(19) United States
(12) Patent Application Publication
(10) Pub. No.: US 2005/0256487 A1
(43) Pub. Date: Nov. 17, 2005

(54) Disposable absorbent article with integral disposal bag

(76) Inventor: Yvette Morton Williams, Piscataway, NJ (US)

Correspondence Address: George A. Frank;DRINKER BIDDLE & REATH LLP, One Logan Square, 18th & Cherry Streets, Philadelphia, PA, 19103-6996, US

(21) Appl. No.: 10/831,419

(22) Filed: Apr. 23, 2004

(51) Int. Cl. A61F 13/15; A61F 13/20

(52) U.S. Cl. 604/385.19; 604/385.23; 604/378

(57) ABSTRACT

A disposable, absorbent article with an integral disposal bag is described. The article includes a moisture permeable layer, a substantially coextensive moisture barrier layer and a moisture absorbent material disposed therebetween. The moisture barrier layer has an opening formed in it. A moisture resistant pouch is disposed between the moisture permeable layer and the moisture barrier layer in communication with the opening. A disposal bag is disposed within the pouch such that the bag is accessible to a user through the opening in the moisture barrier layer. A flap is attached to the exterior surface of the moisture barrier layer and conceals the opening in the moisture barrier layer.

### FIELD OF THE INVENTION

The invention relates to the field of disposable absorbent articles, such as diapers, training pants, adult incontinence products and feminine sanitary napkins.

### BACKGROUND OF THE INVENTION

As many caregivers know, changing a soiled diaper or training pants can be a messy job that often involves unpleasant leaks and pungent odors. Trying to keep the waste contained while changing the diaper or training pants and simultaneously managing an animated baby or child is a challenging and sometimes frustrating project. After the process is complete, the caregiver still must worry about the risk of waste leaking on surrounding objects between the changing area and the disposal receptacle. This problem is of particular concern while changing an overloaded diaper or training pants, or when waste has contaminated the outside of the garment.

When caregivers travel with babies or young children, problems associated with the disposal of diapers or training pants are magnified. There is not always an appropriate place to dispose of used diapers or training pants. Some caregivers may feel inclined to leave soiled garments in inappropriate places instead of properly disposing of them, creating awkward and unsanitary conditions.

The odors associated with soiled diapers or training pants often require that soiled garments be removed from the changing area immediately, regardless of whether or not it is an ideal time to do so. Immediate removal is not usually convenient and may require that the caregiver gather baby to accompany him or her to an outside incinerator or garbage can.

Some caregivers have utilized disposable plastic bags, similar to the type used to transport groceries, to carry and dispose of soiled diapers or training pants. This method requires that, when traveling away from home, the caregiver gather and carry the plastic bags along with spare diapers or training pants and the rest of his or her changing armamentarium. The method is not convenient, and if the plastic bag is forgotten, travel may be particularly challenging and even embarrassing.

Adult incontinence product users face many similar challenges as those described above with regard to the disposal of diapers or training pants. Changing the products and disposing of them can be messy, odorous and embarrassing. Traveling and visiting friends may require that the user leave a soiled garment exposed in the bathroom receptacle, thereby highlighting a potentially embarrassing condition. Public facilities may not have disposal bags available to discreetly seal the garment and allow the user to place it in the provided receptacle.

Disposal of feminine sanitary napkins can present a similar dilemma. Women are usually forced to delicately roll the used napkin and try to wrap it in tissue paper prior to disposal. Some public facilities provide small disposable bags and private receptacles in public stalls; but the practice is not universal. Women may need to carry the used napkin outside the private stall to dispose of it in the general garbage can.

Efforts to alleviate some of the above-described problems include prior attempts to incorporate a disposal bag into a diaper or other article. A recent example is that shown and described in U.S. Pat. App. Pub. No. 2002/0004656 A1, in the name of Khan et al. Khan et al. describe a diaper having coextensive, perforated storage compartments for housing a disposal bag and disposable gloves.

U.S. Pat. App. Pub. No. 2002/0133134 A1, in the name of Wilbon, describes a disposable sack that can be detached from a diaper. The sack can be scented, and can include a drawstring for closing the sack after an absorbent material has been placed therein. The publication indicates that the sack can be used to secure diapers, sanitary napkins and other absorbent materials.

U.S. Pat. No. 4,743,240 to Powell shows another diaper with an integral disposal bag. The Powell diaper includes a plurality of pockets for containing diaper-changing accessories, such as towelettes and powder. The pockets are exposed by a moveable flap. In addition, the Powell diaper includes a bag that can be accessed by a slit in the plastic outer layer of the diaper.

U.S. Pat. No. 5,290,268 to Oliver et al. describes a diaper having a fluid impermeable pocket web for receiving the diaper after it has been soiled. The pocket can include tubular pockets with frangible capsules containing deodorizing fluid to minimize unpleasant odors after use.

These and other references represent improvements in the field of disposable absorbent articles. However, none have completely solved the aforementioned problems with absorbent articles, nor, to the applicant's knowledge, have any enjoyed widespread commercial use. Therefore, a need continues to exist for a disposable absorbent article with an integral disposal bag that alleviates the long standing problems faced by users of such articles or their caregivers.

### SUMMARY OF THE INVENTION

The invention relates to a disposable, absorbent article with an integral disposal bag. The article includes a moisture permeable layer, a substantially coextensive moisture barrier layer and a moisture absorbent material disposed therebetween. The moisture permeable layer and moisture barrier layer have exterior surfaces facing away from the moisture absorbent material and opposed interior surfaces adjacent the moisture absorbent material.

The moisture barrier layer has an opening formed in it. A moisture resistant pouch is disposed between the moisture permeable layer and the moisture barrier layer. The pouch has an open end that can be attached to the interior surface of the moisture barrier layer around the opening in the moisture barrier layer. The pouch can resist migration of moisture from the absorbent material to the interior of the pouch and the opening in the moisture barrier layer.

A disposal bag is disposed within the pouch such that the bag is accessible to a user through the opening in the moisture barrier layer. The disposal bag can be made of a breathable material that allows an adequate amount of airflow to prevent accidental suffocation in the event that the bag is withdrawn by an unsupervised wearer.

A flap is attached to the exterior surface of the moisture barrier layer and conceals the opening in the moisture barrier layer. The flap can be made of a material that is visually similar to the moisture barrier layer and can have a long side and two short sides attached to the exterior surface of the moisture barrier layer.

### BRIEF DESCRIPTION OF THE DRAWINGS

For the purpose of illustrating the invention, there are shown in the drawings forms which are presently preferred; it being understood, that this invention is not limited to the precise arrangements and instrumentalities shown.

FIG. 1 is a rear view of a diaper with an integral disposal bag according to an embodiment of the invention.

FIG. 2 is a view of a disposable pad with an integral disposal bag according to an embodiment of the invention.

FIG. 3 is a rear view of an undergarment with an integral disposal bag according to an embodiment of the invention.

FIG. 4 is a rear view of an adult incontinence product with an integral disposal bag according to an embodiment of the invention.

FIG. 5 is an enlarged view of the portion of the diaper of FIG. 1 that includes the integral disposal bag with a partially upturned closure flap. The elements shown in FIG. 5 are common to the pad of FIG. 2, the undergarment of FIG. 3 and the adult incontinence product of FIG. 4.

FIG. 6 is a cross-sectional view taken along line 6-6 in FIG. 1. The elements shown in FIG. 6 are common to the pad of FIG. 2, the undergarment of FIG. 3 and the adult incontinence product of FIG. 4. As such, FIG. 6 also represents cross-sectional views taken along lines 6A-6A in FIG. 2, 6B-6B in FIGS. 3 and 6C-6C in FIG. 4.

FIG. 7 is a view of the diaper of FIG. 1 with the integral disposal bag withdrawn.

FIG. 8 is a view of the diaper of FIG. 1 with the integral disposal bag withdrawn and unfolded.

### DETAILED DESCRIPTION OF THE DRAWINGS

In the Figures, in which like numerals indicate like elements, there are shown embodiments of a disposable absorbent article with an integral disposal bag. It will be noted that the various embodiments share certain common features, which may be shown and described in detail with reference to a drawing of one such embodiment. It should be understood that, when so indicated, such features are common to the other embodiments.

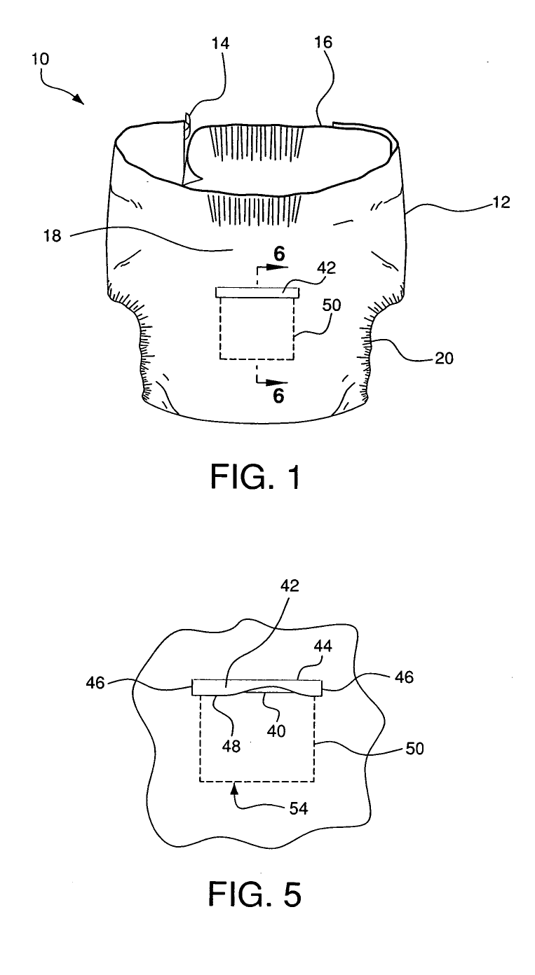

FIG. 1 shows the rear face of a disposable absorbent article according to the present invention embodied as a diaper 10. The diaper 10 is in the form of a substantially rectangular blank 12 having attachment tabs 14 proximate the corners. The diaper generally includes a front portion 16 and a back portion 18 connected by a middle section having elastic leg-engaging areas 20. As depicted in FIG. 1, the back portion 18 is enlarged relative to the front portion 16 so as to more clearly show one of the attachment tabs 14 commonly found in diapers. It will be understood that the front 16 and back 18 portions can instead be of substantially the same width, similar to that shown in U.S. Pat. No. 4,808,175 to Hansen, which is incorporated herein by reference, such that the two portions are affixed together by attachment tabs 14 on the side of the diaper.

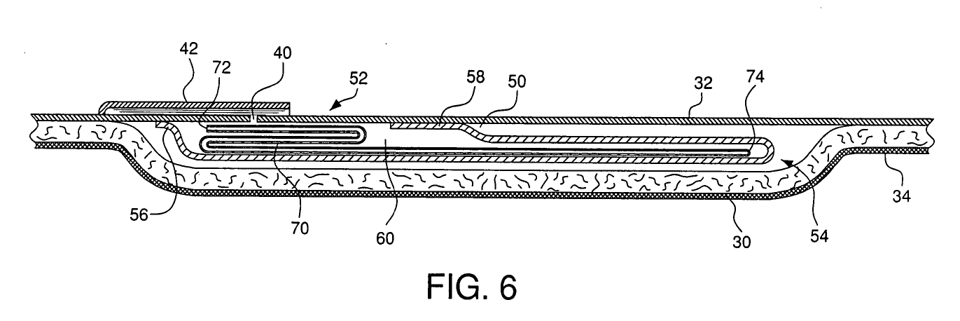

As shown in FIG. 6, the substantially rectangular blank 12 is formed from a moisture permeable layer 30, a substantially coextensive moisture barrier layer 32 and a moisture absorbent material 34 disposed therebetween. The moisture permeable layer 30 and the moisture barrier layer 32 have exterior surfaces facing away from the moisture absorbent material 34 and opposed interior surfaces adjacent the moisture absorbent material 34. In use, the exterior surface of the moisture permeable layer 30 is intended to be on the inside of the diaper in contact with the skin of an infant or other wearer. The exterior surface of the moisture barrier layer 32 is intended to be on the outside of the diaper, away from the skin of an infant or other wearer.

The moisture permeable layer 30 can be a fabric or another material known for making the inside (skin-engaging) layer of a diaper or other absorbent article. Likewise, the moisture barrier layer 32 can be plastic or another moisture impervious material known for making the outside layer of a diaper or other absorbent article. The moisture absorbent material 34 can be any material known in the art for absorbing waste. Any of the layers 30, 32 and material 34 can be formed from a plurality of webs and can include combinations of different materials.

An opening 40 is formed in the moisture barrier layer 32 in the back portion 18 of the diaper 10. A flap 42 covers and conceals the opening 40 from view during conditions of normal use. FIG. 5 is an enlarged view of the area proximate the opening 40, with a portion of the flap 42 pulled away from the opening 40. As seen in FIG. 5, the opening 40 is preferably in the form of a slit in the moisture barrier layer 32.

The flap 42 can be attached to the outside surface of the moisture barrier layer 32. The flap 42 can be substantially rectangular, having a long side 44 and short sides 46 attached to the moisture barrier layer 32. The attached sides 44, 46 can be adhered by means of adhesive, sewn, heat sealed or otherwise affixed to the moisture barrier layer 32. When so configured, the flap 42 is attached to the moisture barrier layer 32 along more than half of the periphery of the flap 42. Thus, when undisturbed, the flap 42 tends to stay flush against the moisture barrier layer 32. The flap 42 can have a free long side 48, which can be manipulated by a user to access the opening 40. The flap 42 can be made of a material that is visually similar to the material of the moisture barrier layer 32. Such a visually similar material can be the material from which the moisture barrier layer 32 is formed.

When formed from a visually similar material as the moisture barrier layer 32, the flap is camouflaged (i.e., visually blended) therewith. In that case, the presence of the flap 42 can be difficult to detect, such that to the casual observer the back portion 18 of the diaper appears to be an uninterrupted piece of material. By camouflaging the flap 42 with the moisture barrier layer 32, casual observers, such as playmates or other associates of the diaper wearer, will likely not take notice of flap 42. As such, casual observers will have little incentive to manipulate the free side 48 of the flap. So long as the flap 42 remains undisturbed, the opening 40 will remain concealed under conditions of normal use. Thus, the chance will be minimized that an observer will investigate the opening 40 or reach through the opening 40 to prematurely withdraw the contents of the pouch, which are described below.

A moisture resistant pouch 50 is disposed between the moisture permeable layer 30 and the moisture barrier layer 32. The pouch 50 has an open end indicated generally in FIG. 6 by the arrow 52 and a closed end indicated by the arrow 54. The open end 52 is attached to the interior surface of the moisture barrier layer 32 around the opening 40 in the moisture barrier layer 32. A first portion 56 of the pouch open end 52 is attached to the interior surface of the moisture barrier layer 32 above the opening 40; and a second portion 58 of the pouch open end 52 is attached to the interior surface of the moisture barrier layer 32 below the opening 40. The length of the opening 40 is shorter than the width of the pouch 50 shown in FIG. 5. The portions of the open end 52 of the pouch 50 that are not shown in section in FIG. 6 are also attached to the interior surface of the moisture barrier layer 32 around the opening 40. Thus, the pouch 50 substantially isolates the opening 40 from the moisture absorbent material 34, such that only the interior of the pouch 50 is in direct fluid communication with the opening 40. The pouch 50 is capable of resisting migration of moisture from the absorbent material 34 to the interior of the pouch 60 and opening 40 in the moisture barrier layer 32. As such, the moisture resistant pouch 50 minimizes the risk of cross-contamination from the moisture absorbent material 34 to the interior of the pouch 50 after the disposable article has been soiled.

A disposal bag 70 is disposed within the pouch 50 and is accessible to a user through the opening 40 in the moisture barrier layer 32. The disposal bag 70 can be folded to fit neatly within the pouch 50 so that a large bulge in the rear portion 18 of the diaper is not formed. Moreover, any actual bulge that is formed should not be noticeable to the casual observer. In FIG. 6, the folds in the disposal bag 70 are shown in a limited number and limited size for clarity. In practice, it is preferred that the disposal bag 70 be folded one or more times in the width dimension and one or more times in the length direction so that the pouch 50 can be made relatively small while still being of adequate size to contain the disposal bag 70. Also, the folded lengths of the disposal bag 70 can take up substantially the entire length of the pouch 50, rather than the shorter folded lengths shown.

The disposal bag includes an open end 72 and a closed end 74. The closed end 74 can be attached to the inside of the pouch 50. The attachment between the closed end of the disposal bag 70 and the pouch 50 is preferably made using adhesive so that the moisture resistance of the pouch 50 is not comprised. However, other means of attachment can instead be used. When attached, the point of attachment can be anywhere within the pouch 50, but is preferably made between a portion of the disposal bag 70 near the closed end 74, and the open end 52 of the pouch 50. The disposal bag 70 can also be attached to the interior surface of the moisture barrier layer 32 within the pouch 50. When so attached, the disposal bag is also attached, albeit indirectly, to the pouch 50. It should be clear that the disposal bag 70 can be folded in any of several configurations within the pouch 50. It should also be clear that the closed end 74 of the disposal bag 70 can be adjacent the open end 52 of the pouch, rather than in the position shown.

The disposal bag 70 can be made of a waterproof or water resistant material. In many embodiments, it is preferred that the material also be resistant to odor transmission. However, the material should be selected according to the circumstances associated with the particular embodiment in which it is incorporated. In the embodiment of FIG. 1, where the disposal bag 70 is an integral portion of the diaper 10, the disposal bag 70 can be a clear plastic material.

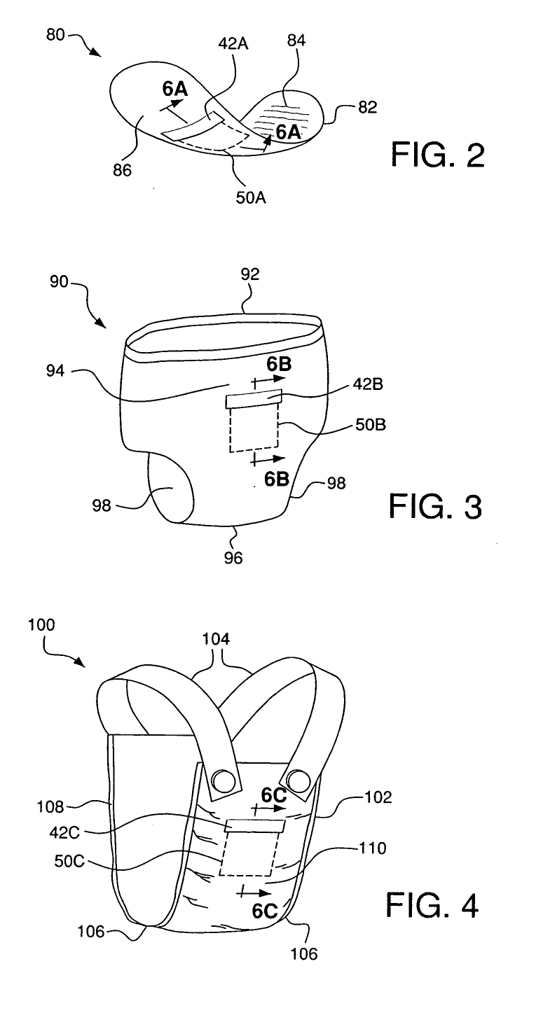

Those elements shown in FIGS. 5 and 6 and described above in connection with the diaper 10 of FIG. 1 are common to the pad 80 of FIG. 2, the undergarment 90 of FIG. 3 and the adult incontinence product 100 of FIG. 4, which are described below. The elements described in connection with the pad 80 that include a reference numeral followed by the designation “A” are analogous to the same numbered element (without the “A” designation) shown in FIGS. 5 and 6. Similarly, the elements described below in connection with the undergarment 90 and adult incontinence product 100 that include a reference numeral followed by the designation “B” or “C”, respectively, are analogous to the same numbered element (without the “B” or “C” designation) shown in FIGS. 5 and 6. Thus, it should be understood that each element shown in FIGS. 5 and 6 also represents the analogous “A”, “B”, and “C”-designated elements of the pad 80, undergarment 90, and adult incontinence product 100, respectively. As such, the descriptions of the common elements set forth above are equally applicable to the analogous “A”, “B”, and “C”-designated elements discussed below.

FIG. 2 shows an embodiment of the present invention in which the disposable absorbent article is a pad 80 in the form of a substantially oval-shaped body 82 with an upper surface 84 and a lower surface 86. The pad body 82 can instead be a substantially rectangular shape. The pad 80 can be any type of personal hygiene pad, such as an adult incontinence product or feminine sanitary napkin.

Consistent with the convention explained above, the cross-sectional line 6A-6A in FIG. 2 represents the relationship of FIG. 6 to FIG. 2. The upper surface 84 of the pad 80 is the moisture permeable layer 30A. The lower surface 86 is the moisture barrier layer 32A. The moisture absorbent material 34A is disposed between the moisture permeable layer 30A and the moisture barrier layer 32A. The moisture barrier layer 32A includes an opening 40A, which, during conditions of normal use, is concealed by the flap 42A disposed on the exterior surface of the moisture barrier layer 32A.

The moisture resistant pouch 50A is disposed between the moisture permeable layer 30A and the moisture barrier layer 32A. The pouch 50A has an open end indicated generally by the arrow 52A and a closed end indicated by the arrow 54A. The open end 52A is attached to the interior surface of the moisture barrier layer 32A around the opening 40A in the manner that has already been described.

The disposal bag 70A is disposed within the pouch 50A and is accessible to a user through the opening 40A in the moisture barrier layer 32A. The disposal bag 70A includes an open end 72A and a closed end 74A. The disposal bag 70A can be folded to fit neatly within the pouch 50A and the closed end 74A can be attached to the inside of the pouch 50A according to the configurations previously described.

The disposal bag 70A is preferably made of an opaque material for discrete disposal of the pad 80. The opaque material can be plastic in order to provide the desired waterproof, water resistant, and/or odor-containing properties.

FIG. 3 shows a disposable absorbent article according to the present invention embodied as an undergarment 90 having a front 92, a back 94, a central crotch area 96 and a pair of leg-engaging openings 98 adjacent the crotch area. In certain forms of the undergarment 90, the leg engaging openings can be defined by elastic leg members (not shown) extending downwardly from the crotch area 96. The undergarment 90 can be an adult incontinence product or training pants, similar to those known as PULL-UPS, a registered trademark of Kimberly-Clark Worldwide, Inc. of Neenah, Wis.

Similar to the “A”-designated elements of the pad 80 in FIG. 2, the undergarment 90 includes those elements shown in FIGS. 5 and 6 that are not labeled in FIG. 3. As previously noted, the common elements shown in FIGS. 5 and 6 will be identified with the designation “B” when referring to those elements as incorporated in the undergarment 90.

The cross-sectional line 6B-6B in FIG. 3 represents the relationship of FIG. 6 to FIG. 3. The undergarment 90 is formed from the moisture permeable layer 30B, the substantially coextensive moisture barrier layer 32B and the moisture absorbent material 34B disposed therebetween. The moisture permeable layer 30B and the moisture barrier layer 32B have exterior surfaces facing away from the moisture absorbent material 34B and opposed interior surfaces adjacent the moisture absorbent material 34B. In use, the exterior surface of the moisture permeable layer 30B is intended to be on the inside of the undergarment 90 in contact with the skin of a wearer. The exterior surface of the moisture barrier layer 32B is intended to be on the outside of the undergarment 90, away from the skin of a wearer. The opening 40B is formed in the moisture barrier layer 32B in the back 94 of the undergarment. The flap 42B covers and conceals the opening 40B from view during conditions of normal use. The flap 42B can be of the same shape and attached in the same way as described above.

The moisture resistant pouch 50B is disposed between the moisture permeable layer 30B and the moisture barrier layer 32B. The pouch 50B has an open end indicated generally by the arrow 52B and a closed end indicated by the arrow 54B. The open end 52B is attached to the interior surface of the moisture barrier layer 32B around the opening 40B in the previously described manner.

The disposal bag 70B is disposed within the pouch 50B and is accessible to a user through the opening 40B in the moisture barrier layer 32B. The disposal bag 70B includes an open end 72B and a closed end 74B. The disposal bag 70B can be folded to fit neatly within the pouch 50B; and the closed end 74B can be attached to the inside of the pouch 50B as described above.

The disposal bag 70B is preferably made of a breathable material that allows an adequate amount of airflow to prevent accidental suffocation. Use of a breathable material for the disposal bag 70B is especially advantageous in the undergarment 90 when the undergarment is used as training pants for a young child. By their inquisitive nature, children tend to investigate their environments, which may include the undergarment the child or the child's playmate may be wearing. There exists a chance that the flap 42B and the concealed opening 40B may become discovered. Upon discovering same, it may be possible that a child could, upon further investigation, withdraw the disposal bag from the pouch 50B while a caregiver is not paying close attention. Making the disposal bag 70B of a breathable material minimizes any risk of accidental suffocation based on what the child does with the disposal bag upon discovery of same.

FIG. 4 shows another embodiment of a disposable absorbent article according the present invention. According to this embodiment, the disposable absorbent article is an adult incontinence product 100 in the form of a substantially rectangular blank 102 having connectors 104 proximate corners of the blank. The product 100 includes leg-engaging areas 106 disposed between a front portion 108 of the adult incontinence product and a back portion 110 of the adult incontinence product. In certain forms for the adult incontinence product 100, the leg engaging areas 106 can be elastic, similar to the leg-engaging areas 20 of the diaper 10.

Similar to the “A”-designated elements of the pad 80 and the “B”-designated elements of the undergarment 90, the adult incontinence product 100 includes those elements shown in FIGS. 5 and 6 that are not labeled in FIG. 4. As noted above, the common elements shown in FIGS. 5 and 6 will be designated with the letter “C” when referring to those elements as incorporated in the adult incontinence product 100.

The cross-sectional line 6C-6C in FIG. 4 represents the relationship of FIG. 6 to FIG. 4. The adult incontinence product 100 is formed from the moisture permeable layer 30C, the substantially coextensive moisture barrier layer 32C and the moisture absorbent material 34C disposed therebetween. The moisture permeable layer 30C and the moisture barrier layer 32C have exterior surfaces facing away from the moisture absorbent material 34C and opposed interior surfaces adjacent the moisture absorbent material 34C. In use, the exterior surface of the moisture permeable layer 30C is intended to be on the inside of the adult incontinence product 100 in contact with the skin of a wearer. The exterior surface of the moisture barrier layer 32C is intended to be on the outside of the adult incontinence product 100, away from the skin of a wearer.

The opening 40C is formed in the moisture barrier layer 32C in the back portion 110 of the adult incontinence product 100. The flap 42C covers and conceals the opening 40C from view during conditions of normal use. The flap 42C can be of the same shape and attached to the moisture barrier layer 32C in the same way as described above.

The moisture resistant pouch 50C is disposed between the moisture permeable layer 30C and the moisture barrier layer 32C. The pouch 50C has an open end indicated generally by the arrow 52C and a closed end indicated by the arrow 54C. The open end 52C is attached to the interior surface of the moisture barrier layer 32C around the opening 40C in the previously described manner.

The disposal bag 70C is disposed within the pouch 50C and is accessible to a user through the opening 40C in the moisture barrier layer 32C. The disposal bag 70C includes an open end 72C and a closed end 74C. The disposal bag 70C can be folded to fit neatly within the pouch 50C; and the closed end 74C can be attached to the inside of the pouch 50C as described above.

The disposal bag 70C is preferably made of an opaque material. Provision of an opaque disposal bag 70C is preferred so that an adult user can discretely dispose of the adult incontinence product 100 after use. The possibility of discrete disposal can make the user feel comfortable in situations where the user finds it necessary to dispose of the product at a friend's residence or other location where people unfamiliar with the product's use may be. The opaque material can be plastic in order to also provide the desired waterproof, water resistant, and/or odor-containing properties.

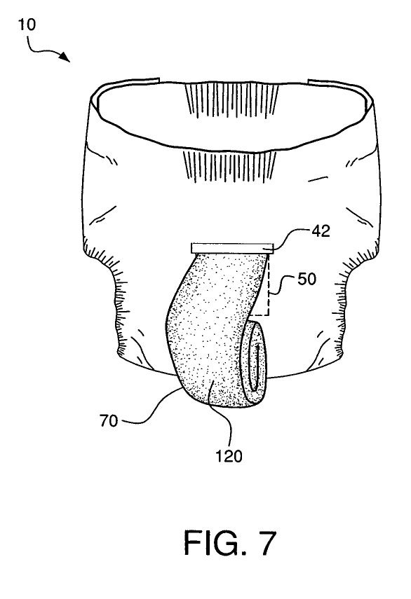

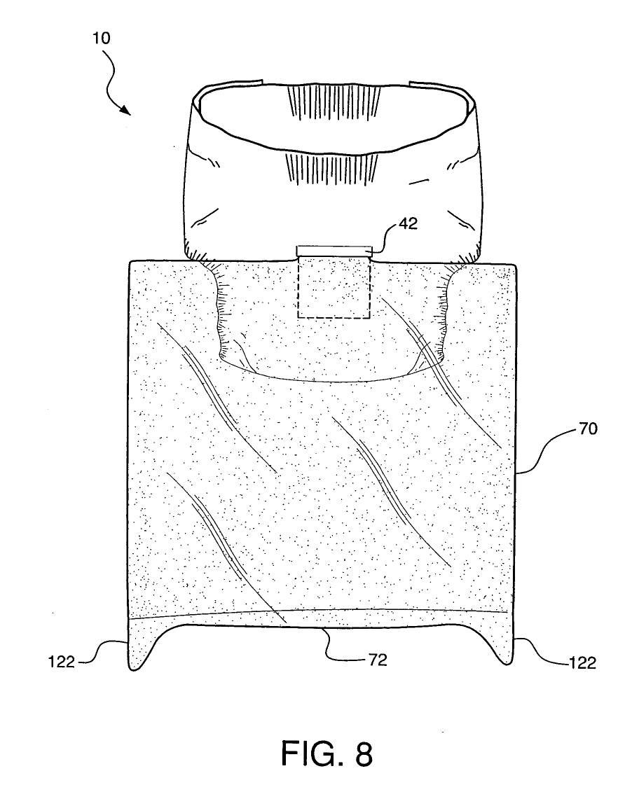

It should now be clear that the disposable absorbent article according of the present invention includes certain common features, regardless of the form in which the article is manifested. Disposal of the absorbent article also involves common steps, regardless of form. As such, disposal of the article will now be explained with reference to all of the embodiments, with the diaper 10 shown as an example in FIGS. 7 and 8.

Once the absorbent article has been soiled and requires disposal, the user, who may be the wearer or a caregiver, can manipulate the free side 48 of the flap 42 to reveal the opening 40. It is also possible to attach the flap 42 to the moisture barrier layer 32 in such a way so that it can be torn away from the moisture barrier layer 32 to assist impaired users who may have trouble manipulating the flap 42. The user can reach into the opening 40, grasp and withdraw the disposal bag 70 from the pouch 50. The withdrawn disposal bag 70 is shown in FIG. 7.

The disposal bag 70 can be provided with a deodorizer and/or a disinfectant 120. The deodorizer or disinfectant can be provided by placing it in the pouch 50 along with the disposal bag 70. The deodorizer or disinfectant can be provided in powder or other suitable form. Some of the deodorizer or disinfectant 120 can stick to the outside of the disposal bag 70 when it is withdrawn from the pouch 50.

After withdrawing the disposal bag 70 from the pouch 50, the disposal bag 70 can be unfolded, as shown in FIG. 8. If the closed end 74 of the disposal bag is attached to the inside of the pouch 50 as described above, the possibility that the bag 70 will separate from the absorbent article is minimized. With the disposal bag 70 withdrawn and unfolded, it can be seen that the disposal bag 70 can have an integral tying feature 122. The integral tying feature can be a pair of tabs extending from the open end 72 of the disposal bag. Such tabs can be similar to those provided on certain known kitchen trash bags to facilitate tying the bag. Alternatively, the integral tying feature can be a draw string, adhesive strip with release liner or other closure feature.

It should also be apparent from FIG. 8 that the disposal bag 70 can be sized appropriately to envelop the disposable article without having excessive unused volume within the disposal bag 70. Therefore, the amount of material used to make the disposal bag can be optimized so that excessive material is not used, while still providing a disposal bag 70 that is capable of fully enveloping the article and then being tied or otherwise securely closed.

Once withdrawn and unfolded, the user can envelop the soiled disposable article in disposal bag 70 by reaching into the open end 72 and grasping the article using the wall of the disposal bag 70 as a barrier between the user's hand and the soiled article. The user can then invert the disposal bag 70 around the article to contain same. The inverting step can be done in similar fashion as that shown and described in U.S. Pat. No. 3,865,110 to Traverse, which is incorporated herein by reference. Once the article is enveloped within the disposal bag 70, the bag 70 can be tied or otherwise sealed.

Once the disposal bag 70 has been inverted, the disinfectant or deodorizer 120 can be on the inside of the disposal bag 70, in contact with the soiled article, thereby minimizing the risk of producing offensive odor and/or decreasing the possibility of handling infectious waste. Also, if an appropriate place for disposal of the article is not readily available, the user can retain the soiled article in the sealed bag 70 without fear of leakage or unpleasant odor until an appropriate disposal place is available.

The present invention may be embodied in other specific forms without departing from the essential attributes thereof. Accordingly, reference should be made to the appended claims, rather than to the foregoing specification, as indicating the scope of the invention.

1. A disposable, absorbent article with an integral disposal bag, the article comprising:
a moisture permeable layer, a substantially coextensive moisture barrier layer and a moisture absorbent material disposed therebetween, the moisture permeable layer and moisture barrier layer having exterior surfaces and opposed interior surfaces;
the moisture barrier layer having an opening formed therein;
a moisture resistant pouch disposed between the moisture permeable layer and the moisture barrier layer, the pouch having an open end and a closed end, the open end being attached to the interior surface of the moisture barrier layer around the opening in the moisture barrier layer, the pouch being capable of resisting migration of moisture from the absorbent material to the interior of the pouch and opening in the moisture barrier layer;
a disposal bag disposed within the pouch, the bag being accessible through the opening in the moisture barrier layer; and
a flap attached to the exterior surface of the moisture barrier layer and concealing the opening in the moisture barrier layer.

2. The disposable, absorbent article of claim 1 further comprising a deodorizer disposed in the pouch.

3. The disposable, absorbent article of claim 1 further comprising a disinfectant disposed in the pouch.

4. The disposable, absorbent article of claim 1 wherein the flap is attached to the moisture barrier layer along more than half of the periphery of the flap.

5. The disposable, absorbent article of claim 4 wherein the opening in the moisture barrier layer is a slit and the flap is substantially rectangular, the flap being attached to the moisture barrier layer along a long side and two short sides of the flap.

6. The disposable, absorbent article of claim 1 wherein the disposal bag comprises an integral tying feature.

7. The disposable, absorbent article of claim 1 wherein the disposal bag comprises a breathable material.

8. The disposable, absorbent article of claim 1 wherein a closed end of the disposal bag is attached to the inside of the pouch.

9. The disposable, absorbent article of claim 1 wherein the disposal bag is opaque.

10. The disposable, absorbent article of claim 9 wherein the article is an adult incontinence product in the form of a substantially rectangular blank and further comprising connectors proximate corners of the blank and leg-engaging areas disposed between a front portion of the adult incontinence product and a back portion of the adult incontinence product, wherein the flap and the opening in the moisture barrier layer are disposed in the back portion.

11. The disposable, absorbent article of claim 1 wherein the article is a diaper in the form of a substantially rectangular blank and further comprising attachment tabs proximate corners of the blank and elastic leg-engaging areas disposed between a front portion of the diaper and a back portion of the diaper, wherein the flap and the opening in the moisture barrier layer are disposed in the back portion.

12. The disposable, absorbent article of claim 1 wherein the article is an undergarment further comprising a front, a back, a central crotch area and a pair of leg-engaging openings adjacent the crotch area and optionally extending downwardly therefrom, wherein the flap and the opening in the moisture barrier layer are disposed in the back.

13. The disposable, absorbent article of claim 1 wherein the article is a pad in the form of a substantially rectangular or substantially oval-shaped body with an upper surface and a lower surface, the upper surface being the moisture permeable layer, wherein the flap and the opening in the moisture barrier layer are disposed on the lower surface.

14. A disposable, absorbent article with an integral disposal bag, the article comprising:
a moisture permeable layer, a substantially coextensive moisture barrier layer and a moisture absorbent material disposed therebetween, the moisture permeable layer and moisture barrier layer having exterior surfaces and opposed interior surfaces;
the moisture barrier layer having a slit formed therein;
a moisture resistant pouch disposed between the moisture permeable layer and the moisture barrier layer, the pouch being open to the slit in the moisture barrier layer;
a disposal bag disposed within the pouch, the bag being accessible through the slit; and
a flap comprising a material that is visually similar to the moisture barrier layer, the flap concealing the slit and having a long side and two short sides attached to the exterior surface of the moisture barrier layer.

15. The disposable, absorbent article of claim 14 wherein the pouch comprises an open end and a closed end, the open end being attached to the interior surface of the moisture barrier layer around the slit, the pouch being capable of resisting migration of moisture from the absorbent material to the interior of the pouch and slit.

16. The disposable, absorbent article of claim 14 further comprising a deodorizer disposed in the pouch.

17. The disposable, absorbent article of claim 14 further comprising a disinfectant disposed in the pouch.

18. The disposable, absorbent article of claim 14 wherein the disposal bag comprises an integral tying feature.

19. The disposable, absorbent article of claim 14 wherein a closed end of the disposal bag is attached to the inside of the pouch.

20. The disposable, absorbent article of claim 14 wherein the disposal bag comprises a breathable material.

21. The disposable, absorbent article of claim 14 wherein the disposal bag is opaque.

22. The disposable, absorbent article of claim 21 wherein the article is an adult incontinence product in the form of a substantially rectangular blank and further comprising connectors proximate corners of the blank and leg-engaging areas disposed between a front portion of the adult incontinence product and a back portion of the adult incontinence product, wherein the slit and flap are disposed in the back portion.

23. The disposable, absorbent article of claim 14 wherein the article is a diaper in the form of a substantially rectangular blank and further comprising attachment tabs proximate corners of the blank and elastic leg-engaging areas disposed between a front portion of the diaper and a back portion of the diaper, wherein the slit and flap are disposed in the back portion.

24. The disposable, absorbent article of claim 14 wherein the article is an undergarment further comprising a front, a back, a central crotch area and a pair of leg-engaging openings adjacent the crotch area and optionally extending downwardly therefrom, wherein the slit and flap are disposed in the back.

25. The disposable, absorbent article of claim 14 wherein the article is a pad in the form of a substantially rectangular or substantially oval-shaped body with an upper surface and a lower surface, the upper surface being the moisture permeable layer, wherein the slit and flap are disposed on the lower surface.

26. A disposable, absorbent article with an integral disposal bag, the article comprising:
a moisture permeable layer, a substantially coextensive moisture barrier layer and a moisture absorbent material disposed therebetween, the moisture permeable layer and moisture barrier layer having exterior surfaces and opposed interior surfaces;
the moisture barrier layer having an opening formed therein;
a moisture resistant pouch disposed between the moisture permeable layer and the moisture barrier layer, the pouch being in communication with the opening in the moisture barrier layer;
a disposal bag disposed within the pouch, the disposal bag being accessible through the opening in the moisture barrier layer and comprising a breathable material that allows an adequate amount of airflow to prevent accidental suffocation; and
a flap for concealing the opening in the moisture barrier layer, the flap being attached to the exterior surface of the moisture barrier layer.

27. The disposable, absorbent article of claim 26 further comprising a deodorizer disposed in the pouch.

28. The disposable, absorbent article of claim 26 further comprising a disinfectant disposed in the pouch.

29. The disposable, absorbent article of claim 26 wherein the opening in the moisture barrier layer is a slit and the flap is substantially rectangular, the flap being attached to the moisture barrier layer along a long side and two short sides.

30. The disposable, absorbent article of claim 26 wherein the disposal bag comprises an integral tying feature.

31. The disposable, absorbent article of claim 26 wherein a closed end of the disposal bag is attached to the inside of the pouch.

32. The disposable, absorbent article of claim 26 wherein the pouch comprises an open end and a closed end, the open end being attached to the interior surface of the moisture barrier layer around the opening in the moisture barrier layer, the pouch being capable of resisting migration of moisture from the absorbent material to the interior of the pouch and the opening in the moisture barrier layer.

33. The disposable, absorbent article of claim 26 wherein the disposal bag is opaque.

34. The disposable, absorbent article of claim 33 wherein the article is an adult incontinence product in the form of a substantially rectangular blank and further comprising connectors proximate corners of the blank and leg-engaging areas disposed between a front portion of the adult incontinence product and a back portion of the adult incontinence product, wherein the flap and the opening in the moisture barrier layer are disposed in the back portion.

35. The disposable, absorbent article of claim 26 wherein the article is a diaper in the form of a substantially rectangular blank and further comprising attachment tabs proximate corners of the blank and elastic leg-engaging areas disposed between a front portion of the diaper and a back portion of the diaper, wherein the flap and opening in the moisture barrier layer are disposed in the back portion.

36. The disposable, absorbent article of claim 26 wherein the article is an undergarment further comprising a front, a back, a central crotch area and a pair of leg-engaging openings adjacent the crotch area and optionally extending downwardly therefrom, wherein the flap and opening in the moisture barrier layer are disposed in the back.

37. The disposable, absorbent article of claim 26 wherein the article is a pad in the form of a substantially rectangular or substantially oval-shaped body with an upper surface and a lower surface, the upper surface being the moisture permeable layer, wherein the flap and opening in the moisture barrier layer are disposed on the lower surface.
